SEQUENCE LISTING (53 sequences)

SEQ ID NO: 1 - DNA, 27 bp, Artificial Sequence
  Description of Artificial Sequence Synthetic probe
ggtccttcatcgctcagccttcaccgg

SEQ ID NO: 2 - DNA, 27 bp, Artificial Sequence
  Description of Artificial Sequence Synthetic oligonucleotide
ggtccttcatcgctcagccatcaccgg

SEQ ID NO: 3 - DNA, 27 bp, Artificial Sequence
  Description of Artificial Sequence Synthetic oligonucleotide
ggtccttcatccctcagccttcaccgg

SEQ ID NO: 4 - DNA, 27 bp, Artificial Sequence
  Description of Artificial Sequence Synthetic oligonucleotide
ggtccttaatcgctcagccatcaccgg

SEQ ID NO: 5 - DNA, 27 bp, Artificial Sequence
  Description of Artificial Sequence Synthetic oligonucleotide
ggtccttaatcgctcaggcttcaccgg

SEQ ID NO: 6 - DNA, 27 bp, Artificial Sequence
  Description of Artificial Sequence Synthetic oligonucleotide
ggtccatcatccctcagccttcatcgg

SEQ ID NO: 7 - DNA, 27 bp, Artificial Sequence
  Description of Artificial Sequence Synthetic oligonucleotide
ggtccttgatcggtcagccatcaccgg

SEQ ID NO: 8 - DNA, 21 bp, Artificial Sequence
  Description of Artificial Sequence Synthetic primer
gcccagcaagatccaatctca

SEQ ID NO: 9 - DNA, 22 bp, Artificial Sequence
  Description of Artificial Sequence Synthetic primer
gtggcagggcgctacgaacaat

SEQ ID NO: 10 - DNA, 22 bp, Artificial Sequence
  Description of Artificial Sequence Synthetic primer
gaaaaaacaatagaagaattat

SEQ ID NO: 11 - DNA, 22 bp, Artificial Sequence
  Description of Artificial Sequence Synthetic primer
tgcttttttgaattcttcttta

SEQ ID NO: 12 - DNA, 23 bp, Artificial Sequence
  Description of Artificial Sequence Synthetic primer
gtcggtacacgatattcttcacg

SEQ ID NO: 13 - DNA, 24 bp, Artificial Sequence
  Description of Artificial Sequence Synthetic primer
gagatttcagtagataatacaaca

SEQ ID NO: 14 - DNA, 22 bp, Artificial Sequence
  Description of Artificial Sequence Synthetic primer
atttgaagaggttgcaaacgat

SEQ ID NO: 15 - DNA, 25 bp, Artificial Sequence
  Description of Artificial Sequence Synthetic primer
ttcactctgaagttttcttgtgttc

SEQ ID NO: 16 - DNA, 20 bp, Artificial Sequence
  Description of Artificial Sequence Synthetic primer
taatgctttgatcggccttg

SEQ ID NO: 17 - DNA, 20 bp, Artificial Sequence
  Description of Artificial Sequence Synthetic primer
tggattgcacttcatcttgg

SEQ ID NO: 18 - DNA, 27 bp, Artificial Sequence
  Description of Artificial Sequence Synthetic primer
atgaacaacgttctgaaattctctgct

SEQ ID NO: 19 - DNA, 21 bp, Artificial Sequence
  Description of Artificial Sequence Synthetic primer
cttgcggctggctttttccag

SEQ ID NO: 20 - DNA, 20 bp, Artificial Sequence
  Description of Artificial Sequence Synthetic primer
tatggaatttcgccgatttt

SEQ ID NO: 21 - DNA, 20 bp, Artificial Sequence
  Description of Artificial Sequence Synthetic primer
tgtttgcctccctgctgcgg

SEQ ID NO: 22 - DNA, 70 bp, Artificial Sequence
  Description of Artificial Sequence Synthetic oligonucleotide
gcccagcaagatccaatctcaggtccttcatcgctcagccatcaccgggaaaaaacaatagaagaattat

SEQ ID NO: 23 - DNA, 44 bp, Artificial Sequence
  Description of Artificial Sequence Synthetic oligonucleotide
gtggcagggcgctacgaacaattgcttttttgaattcttcttta

SEQ ID NO: 24 - DNA, 71 bp, Artificial Sequence
  Description of Artificial Sequence Synthetic oligonucleotide
gcccagcaagatccaatctcaggtccttcatccctcagccttcaccgggtcggtacacgatattcttcacg

SEQ ID NO: 25 - DNA, 46 bp, Artificial Sequence
  Description of Artificial Sequence Synthetic oligonucleotide
gtggcagggcgctacgaacaatgagatttcagtagataatacaaca

SEQ ID NO: 26 - DNA, 70 bp, Artificial Sequence
  Description of Artificial Sequence Synthetic oligonucleotide
gcccagcaagatccaatctcaggtccttaatcgctcagccatcaccggatttgaagaggttgcaaacgat

SEQ ID NO: 27 - DNA, 47 bp, Artificial Sequence
  Description of Artificial Sequence Synthetic oligonucleotide
gtggcagggcgctacgaacaatttcactctgaagttttcttgtgttc

SEQ ID NO: 28 - DNA, 68 bp, Artificial Sequence
  Description of Artificial Sequence Synthetic oligonucleotide
gcccagcaagatccaatctcaggtccttaatcgctcaggcttcaccggtaatgctttgatcggccttg

SEQ ID NO: 29 - DNA, 42 bp, Artificial Sequence
  Description of Artificial Sequence Synthetic oligonucleotide
gtggcagggcgctacgaacaattggattgcacttcatcttgg

SEQ ID NO: 30 - DNA, 75 bp, Artificial Sequence
  Description of Artificial Sequence Synthetic oligonucleotide
gcccagcaagatccaatctcaggtccatcatccctcagccttcatcggatgaacaacgttctgaaattctctgct

SEQ ID NO: 31 - DNA, 43 bp, Artificial Sequence
  Description of Artificial Sequence Synthetic oligonucleotide
gtggcagggcgctacgaacaatcttgcggctggctttttccag

SEQ ID NO: 32 - DNA, 68 bp, Artificial Sequence
  Description of Artificial Sequence Synthetic oligonucleotide
gcccagcaagatccaatctcaggtccttgatcggtcagccatcaccggtatggaatttcgccgatttt

SEQ ID NO: 33 - DNA, 42 bp, Artificial Sequence
  Description of Artificial Sequence Synthetic oligonucleotide
gtggcagggcgctacgaacaattgtttgcctccctgctgcgg

SEQ ID NO: 34 - DNA, 18 bp, Artificial Sequence
  Description of Artificial Sequence Synthetic primer
ttgcgccatacggacgat

SEQ ID NO: 35 - DNA, 22 bp, Artificial Sequence
  Description of Artificial Sequence Synthetic primer
gcgacgtcatcggtaaatacca

SEQ ID NO: 36 - DNA, 17 bp, Artificial Sequence
  Description of Artificial Sequence Synthetic primer
tgagccatgcgcaccat

SEQ ID NO: 37 - DNA, 23 bp, Artificial Sequence
  Description of Artificial Sequence Synthetic primer
ggcgagattttgggtaaatacca

SEQ ID NO: 38 - DNA, 16 bp, Artificial Sequence
  Description of Artificial Sequence Synthetic primer
cccccatggcgagaaa

SEQ ID NO: 39 - DNA, 25 bp, Artificial Sequence
  Description of Artificial Sequence Synthetic primer
gttaggcagacggcttggtaraata

SEQ ID NO: 40 - DNA, 16 bp, Artificial Sequence
  Description of Artificial Sequence Synthetic primer
cccccatggcgagaag

SEQ ID NO: 41 - DNA, 25 bp, Artificial Sequence
  Description of Artificial Sequence Synthetic primer
gttaggcagacggcttggtaraata

SEQ ID NO: 42 - DNA, 22 bp, Artificial Sequence
  Description of Artificial Sequence Synthetic primer
cggaaaacgcctacttaaacta

SEQ ID NO: 43 - DNA, 19 bp, Artificial Sequence
  Description of Artificial Sequence Synthetic primer
gtgccgttaagcaaaatgt

SEQ ID NO: 44 - DNA, 22 bp, Artificial Sequence
  Description of Artificial Sequence Synthetic primer
cggaaaacgcctacttaaactt

SEQ ID NO: 45 - DNA, 19 bp, Artificial Sequence
  Description of Artificial Sequence Synthetic primer
gtgccgttaagcaaaatgt

SEQ ID NO: 46 - DNA, 64 bp, Artificial Sequence
  Description of Artificial Sequence Synthetic oligonucleotide
gcccagcaagatccaatctcaggtccttcatcgctcagccatcaccggcccccatggcgagaaa

SEQ ID NO: 47 - DNA, 47 bp, Artificial Sequence
  Description of Artificial Sequence Synthetic oligonucleotide
gtggcagggcgctacgaacaatgttaggcagacggcttggtaraata

SEQ ID NO: 48 - DNA, 64 bp, Artificial Sequence
  Description of Artificial Sequence Synthetic oligonucleotide
gcccagcaagatccaatctcaggtccttcatccctcagccttcaccggcccccatggcgagaag

SEQ ID NO: 49 - DNA, 47 bp, Artificial Sequence; identical to SEQ ID NO: 47
  Description of Artificial Sequence Synthetic oligonucleotide

SEQ ID NO: 50 - DNA, 70 bp, Artificial Sequence
  Description of Artificial Sequence Synthetic oligonucleotide
gcccagcaagatccaatctcaggtccttaatcgctcagccatcaccggcggaaaacgcctacttaaacta

SEQ ID NO: 51 - DNA, 41 bp, Artificial Sequence
  Description of Artificial Sequence Synthetic oligonucleotide
gtggcagggcgctacgaacaatgtgccgttaagcaaaatgt

SEQ ID NO: 52 - DNA, 70 bp, Artificial Sequence
  Description of Artificial Sequence Synthetic oligonucleotide
gcccagcaagatccaatctcaggtccttaatcgctcaggcttcaccggcggaaaacgcctacttaaactt

SEQ ID NO: 53 - DNA, 41 bp, Artificial Sequence; identical to SEQ ID NO: 51
  Description of Artificial Sequence Synthetic oligonucleotide

(19) United States
(12) Patent Application Publication
(10) Pub. No.: US 2021/0269868 A1
(43) Pub. Date: Sep. 2, 2021

(54) PCR INCORPORATION OF EXOGENOUS NUCLEIC ACID SEQUENCES ENABLING MELT-BASED DETECTION

(71) Applicant: CFD Research Corporation, Huntsville, AL (US)

(72) Inventors: David Edwin Gaddes, III, Madison, AL (US); Ketan Harendrakumar Bhatt, Huntsville, AL (US); Balabhaskar Prabhakarpandian, Madison, AL (US)

(21) Appl. No.: 17/187,267

(22) Filed: Feb. 26, 2021

Related U.S. Application Data
(60) Provisional application No. 62/983,478, filed on Feb. 28, 2020.

(51) Int. Cl. C12Q 1/6853; C12Q 1/6832; C12Q 1/6816

(52) U.S. Cl. CPC C12Q 1/6853; C12Q 1/6816; C12Q 1/6832

(57) ABSTRACT

A primer composition for amplifying a target nucleic acid of a plurality of target organisms can include: a first primer having in order: 3′-a first target specific sequence; a melt key region; and a first super primer region, wherein the first target specific sequence includes a nucleic acid sequence that specifically hybridizes with a first unique nucleic acid sequence of a first target organism over nucleic acid sequences of other organisms, the melt key region includes a nucleic acid sequence that has less than full identity with a melt key probe and a complement of the melt key region hybridizes with the melt key probe with at least one nucleotide mismatch with the melt key probe, and the first super primer region includes a nucleic acid sequence that is common to the plurality of target organisms.

### CROSS-REFERENCE TO RELATED APPLICATIONS

This patent application claims priority to U.S. Provisional Application No. 62/983,478 filed Feb. 28, 2020, which provisional is incorporated herein by specific reference in its entirety.

### SEQUENCE LISTING

The instant application contains a Sequence Listing which has been submitted electronically in ASCII format and is hereby incorporated by reference in its entirety. Said ASCII copy, created Apr. 21, 2021, is named C1478_10035 US02.SL.txt and is 13, 307 bytes in size.

### BACKGROUND

### Field

The present disclosure relates to systems and methods for amplifying target nucleic acids for use in melt-based detection.

### Description of Related Art

Previously, detection of nucleic acid sequences has been performed by multiplex ligation-dependent probe amplification (MLPA) technique (J. P. Schouten, C. J. McElgunn, R. Waaijer, D. Zwijnenburg, F. Diepvens, and G. Pals, “Relative quantification of 40 nucleic acid sequences by multiplex ligation-dependent probe amplification,” Nucleic acids research, vol. 30, no. 12, pp. e57-e57, 2002). It was found that the design and preparation of MLPA probe mixture was time-consuming and difficult. Later, it was found that multiplex analytical systems allow detection of multiple nucleic acid targets in one assay, which can provide rapid characterization. The combined use of fluorescence color and melting temperature as a virtual 2D label enables homogenous detection of multiple targets (Y. Liao, X. Wang, C. Sha, Z. Xia, Q. Huang, and Q. Li, “Combination of fluorescence color and melting temperature as a two-dimensional label for homogeneous multiplex PCR detection,” Nucleic acids research, vol. 41, no. 7, pp. e76-e76, 2013). More recently, multiplex ligation reaction based on a probe melting curve analysis has been used for simultaneous identification of multiple bacterial pathogens (Y. Jiang, L. He, P. Wu, X. Shi, M. Jiang, Y. Li, Y. Lin, Y. Qiu, F. Bai, and Y. Liao, “Simultaneous identification of ten bacterial pathogens using the multiplex ligation reaction based on the probe melting curve analysis,” Scientific reports, vol. 7, no. 1, p. 5902, 2017). However, there is still a need for improved multiplex nucleic acid amplification and detection, such as improved assay protocols and reduced assay time to obtain results.

Urinary tract infections are among the most common bacterial infections treated in hospitals, as well as in outpatient facilities. However, resistance to the first-line oral antimicrobials used to treat UTIs complicates both outpatient and in-hospital treatment. Antimicrobial-resistant infections are increasingly common, and need rapid diagnostic instrumentation to rapidly and accurately detect the specific bacteria in an infection in order to reduce the overuse of antibiotics and to distribute the most effective antibiotics when an infection is present. Specifically, a need exists to 1) combat a massive overdosing of patients and 2) address the increased difficulty of outpatient treatment due to a rapid rise in multidrug resistance in UTIs. This may be accomplished by nucleic acid detection of the microbes with a fast detection system. Bacterial culture is the standard method to validate an infection and determine antimicrobial-resistance. However, this technique takes 24-72 hours to complete during which time antibiotics will be prescribed to patients prior to the result. A diagnostic tool that can provide a result prior to the distribution of antibiotics as well as informing the selection of antibiotics would be helpful.

Historically, antimicrobial resistance has been addressed through the development of new classes of antimicrobials, however, the development of new and effective treatments has stalled. Therefore, choosing appropriate antimicrobial agents and reducing the frequent overuse of antibiotics is increasingly important. Diagnostic instruments utilizing nucleic acid amplification tests (NAAT) have the potential to provide guidance for the appropriate antibiotic and prevent erroneous prescriptions.

A urine culture is the standard for the diagnosis of UTI and susceptibility testing constituting the vast majority of diagnoses. Although, CLIA laboratory-based NAAT performed by clinical laboratory technicians are available from companies such as Diatherix, which uses a 14-target panel and includes genetic susceptibility information. However, both of these methods require processing a collected urine sample at an external facility, which takes too long to provide actionable information in a POC environment. There are several established POC platforms to perform bacterial identification, such as Cepheid and Biofire. However, the Cepheid GeneXpert is not capable of identifying a large number of bacterial species required for UTI. While the turnaround time of a Biofire Filmarray panel takes 2-hours to compete, which is longer than the 40-minute time frame most women are typically willing to wait in an outpatient facility for test results. In addition, neither Cepheid nor Biofire have developed a panel for UTIs. The current state of diagnostic tools for UTIs is summarized in the table below

TABLE 1
Aggregation of current and potential diagnostics for UTIs

| | Sample-to- | | Number of | Available |
|---|---|---|---|---|
| Diagnostic | Answer Time | | PCR Targets | for UTIs |
| Urine Culture | 24-72 | hours | N/A | Yes |
| Diatherix | >6 | hours | 14 | Yes |
| (TEM-PCR) | | | | |
| Cepheid | >1 | hour | 6 | No |
| GeneXpert | | | | |
| Biofire FilmArray | >2 | hours | 27 | No |

Thus, it would be advantageous to have a technology that can be used for a one pot nucleic acid amplification test (NAAT) that utilizes a PCR process to use exogenous nucleic acid sequences for rapid multiplex nucleic acid amplification and melt-based detection. The target nucleotides may be from bacteria pathogens, and thereby the accurate detection can identify the bacteria pathogens that are present so that the correct antibiotic and dosage can be determined.

### SUMMARY

In some embodiments, a primer composition for amplifying a target nucleic acid of a plurality of target organisms can include: a first primer having in order: 3′-a first target specific sequence; a melt key region; and a first super primer region, wherein the first target specific sequence includes a nucleic acid sequence that specifically hybridizes with a first unique nucleic acid sequence of a first target organism over nucleic acid sequences of other organisms, the melt key region includes a nucleic acid sequence that has full identity or less than full identity with a melt key probe and a complement of the melt key region hybridizes with the melt key probe with no mismatch or at least one nucleotide mismatch with the melt key probe, and the first super primer region includes a nucleic acid sequence that is exogenous to the plurality of target organisms; and a second primer having in order: 3′-target specific sequence; and a second super primer region, wherein the second target specific sequence includes a nucleic acid sequence that specifically hybridizes with a second unique nucleic acid sequence of the first target organism over nucleic acid sequences of other organisms, and the second super primer region includes a nucleic acid sequence that is exogenous to the plurality of target organisms, wherein the first primer and second primer form a primer pair.

In some embodiments, a primer composition for amplifying at least one target nucleic acid of a plurality of target nucleic acids can include: a first primer having in order: 3′-a first target specific sequence; a melt key region; and a first super primer region, wherein the first target specific sequence includes a nucleic acid sequence that specifically hybridizes with a first unique nucleic acid sequence of a first target nucleic acid over nucleic acid sequences of other nucleic acids, the melt key region includes a nucleic acid sequence that has full identity or less than full identity with a melt key probe and a complement of the melt key region hybridizes with the melt key probe with no mismatch or at least one nucleotide mismatch with the melt key probe, and the first super primer region includes a nucleic acid sequence that is exogenous to the plurality of target nucleic acids; and a second primer having in order: 3′-target specific sequence; and a second super primer region, wherein the second target specific sequence includes a nucleic acid sequence that specifically hybridizes with a second unique nucleic acid sequence of the first target nucleic acid over nucleic acid sequences of other nucleic acids, and the second super primer region includes a nucleic acid sequence that is exogenous to the plurality of target nucleic acids, wherein the first primer and second primer form a primer pair.

In some embodiments, the primer composition can include: a plurality of first primers, wherein each first primer has the first target specific sequence that specifically hybridizes with a first nucleic acid sequence of a respective first target organism over nucleic acid sequences of other target organisms, each first primer has a melt key region that is different from melt key regions of other first primers, and each first primer has the same first super primer region; and a plurality of second primers, wherein each second primer has the second target specific sequence that specifically hybridizes with a second nucleic acid sequence of a respective first target organism over nucleic acid sequences of other target organisms, and each second primer has the same second super primer region.

In some embodiments, the primer composition can include: a plurality of first primers, wherein each first primer has the first target specific sequence that specifically hybridizes with a first nucleic acid sequence of a respective first target nucleic acid over nucleic acid sequences of other target nucleic acids, each first primer has a melt key region that is different from melt key regions of other first primers, and each first primer has the same first super primer region; and a plurality of second primers, wherein each second primer has the second target specific sequence that specifically hybridizes with a second nucleic acid sequence of a respective first target nucleic acid over nucleic acid sequences of other target nucleic acids, and each second primer has the same second super primer region.

In some embodiments, the primer composition can include: a first super primer having the sequence of the first super primer region; and a second super primer having the sequence of the second super primer region.

In some embodiments, the primer composition can include the melt key probe, wherein a complement nucleic acid of each melting key region has a different melting temperature with the melt key probe, wherein one first primer can have a melt key region that has the same sequence as the melt key probe and/or each other first primer has a different nucleotide mismatch in the melt key region from each other first primer.

In some embodiments, a melt-based nucleic acid system can include: a melt probe having a nucleotide sequence; and a plurality of first primers, each first primer having in order: 3′-a first target specific sequence; a melt key region; and a first super primer region, wherein the first target specific sequence includes a nucleic acid sequence that specifically hybridizes with a first unique nucleic acid sequence of a first target organism over nucleic acid sequences of other target organisms, the melt key region includes a nucleic acid sequence that has less than full identity (e.g., less than full complementarity, such as 1, 2, 3, or more mismatches) with the melt key probe and a complement of the melt key region (e.g., full identity complementarity) hybridizes with the melt key probe with at least one nucleotide mismatch with the melt key probe, wherein each first primer has a melt key region that is different from melt key regions of other first primers, and each first primer has the same first super primer region; and a plurality of primer nucleic acids each having a melt key region with a nucleotide sequence with less than full uniformity (e.g., less than full identify complementarity, such as 1, 2, 3 or more mismatches) compared to the melt probe, wherein each first primer has a different nucleotide mismatch from each other first primer.

In some embodiments, a melt-based nucleic acid system can include: a melt probe having a nucleotide sequence; and a plurality of first primers, each first primer having in order: 3′-a first target specific sequence; a melt key region; and a first super primer region, wherein the first target specific sequence includes a nucleic acid sequence that specifically hybridizes with a first unique nucleic acid sequence of a first target nucleic acid over nucleic acid sequences of other target nucleic acids, the melt key region includes a nucleic acid sequence that has full identify or less than full identity (e.g., less than full complementarity, such as 1, 2, 3, or more mismatches) with the melt key probe and a complement of the melt key region (e.g., full identity complementarity) hybridizes with the melt key probe with no mismatch or at least one nucleotide mismatch with the melt key probe, wherein each first primer has a melt key region that is different from melt key regions of other first primers, and each first primer has the same first super primer region; and a plurality of primer nucleic acids each having a melt key region with a nucleotide sequence with full uniformity or less than full uniformity (e.g., less than full identify complementarity, such as 1, 2, 3 or more mismatches) compared to the melt probe, wherein each first primer has a different nucleotide mismatch from each other first primer.

In some embodiments, a method of detecting a plurality of target organisms in a sample can include: providing the sample into a PCR device; providing a PCR composition into the PCR device with the sample; performing PCR to generate a plurality of amplicons, each amplicon including one melt key complement region having a nucleotide sequence with full complementarity to the respective melt key region; performing a melt curve analysis with the plurality of amplicons and the melt key probe; detecting at least one melting temperature for the melt key probe and at least one amplicon including one melt key region; and determining presence of at least one target organism of the plurality of target organisms by detecting the presence of a predefined melting temperature for the melt key probe and the at least one amplicon including one melt key region.

In some embodiments, a method of detecting a plurality of target nucleic acids in a sample can include: providing the sample of nucleic acids into a PCR device; providing a PCR composition into the PCR device with the sample; performing PCR to generate a plurality of amplicons, each amplicon including one melt key complement region having a nucleotide sequence with full complementarity to the respective melt key region; performing a melt curve analysis with the plurality of amplicons and the melt key probe; detecting at least one melting temperature for the melt key probe and at least one amplicon including one melt key region; and determining presence of at least one target nucleic acid of the plurality of target nucleic acids by detecting the presence of a predefined melting temperature for the melt key probe and the at least one amplicon including one melt key region.

In some embodiments, the PCR composition can include: a plurality of first primers, each first primer having in order: 3′-a first target specific sequence; a melt key region; and a first super primer region, wherein the first target specific sequence includes a nucleic acid sequence that specifically hybridizes with a first unique nucleic acid sequence of a first target organism over nucleic acid sequences of other target organisms, the melt key region includes a nucleic acid sequence that has less than full identity with a melt key probe and a complement of the melt key region hybridizes with the melt key probe with no nucleotide mismatches or at least one nucleotide mismatch with the melt key probe, and the first super primer region includes a nucleic acid sequence that is exogenous to the plurality of target organisms, wherein each first primer has a melt key region that is different from melt key regions of other first primers, and each first primer has the same first super primer region; a plurality of second primers, each second primer having in order: 3′-target specific sequence; and a second super primer region, wherein the second target specific sequence includes a nucleic acid sequence that specifically hybridizes with a second nucleic acid sequence of the first target organism over nucleic acid sequences of other target organisms, and the second super primer region includes a nucleic acid sequences that is exogenous to the plurality of target organisms, wherein the first primer and second primer form a primer pair; a first super primer having the sequence of the first super primer region; a second super primer having the sequence of the second super primer region; and the melt key probe, wherein each first primer has a different melting temperature with the melt key probe, wherein each first primer has a different nucleotide mismatch from each other first primer.

In some embodiments, the PCR composition can include: a plurality of first primers, each first primer having in order: 3′-a first target specific sequence; a melt key region; and a first super primer region, wherein the first target specific sequence includes a nucleic acid sequence that specifically hybridizes with a first unique nucleic acid sequence of a first target nucleic acid over nucleic acid sequences of other target nucleic acids, the melt key region includes a nucleic acid sequence that has full identity or less than full identity with a melt key probe and a complement of the melt key region hybridizes with the melt key probe with no nucleotide mismatches or at least one nucleotide mismatch with the melt key probe, and the first super primer region includes a nucleic acid sequence that is exogenous to the plurality of target nucleic acids, wherein each first primer has a melt key region that is different from melt key regions of other first primers, and each first primer has the same first super primer region; a plurality of second primers, each second primer having in order: 3′-target specific sequence; and a second super primer region, wherein the second target specific sequence includes a nucleic acid sequence that specifically hybridizes with a second nucleic acid sequence of the first target nucleic acid over nucleic acid sequences of other target nucleic acids, and the second super primer region includes a nucleic acid sequences that is exogenous to the plurality of target organisms, wherein the first primer and second primer form a primer pair; a first super primer having the sequence of the first super primer region; a second super primer having the sequence of the second super primer region; and the melt key probe, wherein each first primer has a different melting temperature with the melt key probe, wherein each first primer has a different nucleotide mismatch from each other first primer.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 illustrates an embodiment of a PCR composition and method of amplification and detection. FIG. 1 discloses SEQ ID NO: 1.

FIG. 2 illustrates another embodiment of a PCR composition and method of amplification and detection. FIG. 2 discloses SEQ ID NO: 1.

FIG. 3 illustrates an embodiment of a melting key probe sequence and corresponding melting key region sequences, the nucleic acid sequences illustrated have sequence identification numbers as shown in Table 2. FIG. 3 discloses SEQ ID NOS: 1-7 respectively, in order of appearance.

FIG. 4 illustrates an graph of a melting curve analysis that shows the melting temperatures for the melting key probe with the corresponding melting key region sequences.

The elements and components in the figures can be arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting.

Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present technology relates to novel primers and primer combinations for use in a one pot NAAT for amplification and melt-based detection of target nucleic acids, such as those of bacteria in an UTI. The novel primers and primer combinations allows for multiplex amplification and detection of a plurality of microbes in an infection, in order to determine the presence or absence of specific microbes. The primers and primer combinations can be used in a PCR system that enables fast sample-to-answer processing times through small sample volumes, rapid thermocycling and a PCR multiplexing assay. The PCR multiplexing assay identifies the target nucleic acids, such as those of a bacterial infection (e.g., UTI). The combinations of primers can be used for both detecting the infection and detecting common susceptibility or resistance markers. The instrument and assay can test for large panels of infectious agents simultaneously.

While the present technology is described as being able to detect organisms in a sample, the technology may also be used to detect different nucleic acids in a sample whether or not from different organisms. For example, the technology can be used to detect different nucleic acids of a same organism. Thus, the detection of organisms described herein can be generally applied to detection of different nucleic acids.

The primers and primer combinations can include a panel for common bacterial species involved in UTIs. However, less common bacterial species as well as additional targets to identify antimicrobial susceptibility or resistance may be included and used. Moreover, a magnetic bead-based purification protocol can be used to extract DNA from a few milliliters of urine and elute the bacterial DNA in 20 microliters of PCR solution. From assay start time with the sample until the outcome answer as whether the target nucleic acids is present in the sample can be as low as 30 minutes.

The primer combinations can be adapted for a PCR assay to detect the pathogens Enterococcus faecium, Staphylococcus aureus, Klebsiella pneumoniae, Acinetobacter baumannii, Pseudomonas aeruginosa, and Escherichia coli (e.g., ESKAPE) using a single fluorescence channel with a standard qPCR thermal cycler. However, a dPCR system may be used. In some aspects, the primer combinations include target primer pair for each target bacteria. In some aspects, the primer combinations include an outer primer pair and inner primer pair that can be used in a nested PCR protocol. The threshold cycle of the nested PCR can be compared against that of the outer primers alone to determine whether the single pair or outer primer and inner primer combinations are useful. The detection of the six ESKAPE bacteria in one fluorescence channel by melt-based detection of amplified nucleic acids as described herein.

These six ESKAPE pathogens are the leading cause of nosocomial infections and account for 31.1% of UTIs. Specifically, E. coli, Klebsiella pneumoniae, and Pseudomonas aeruginosa are commonly the cause of UTIs and are increasingly resistant to first-line antibiotics. However, conventional detection of UTIs via bacterial culture can take between 24 and 72 hours, at which point antibiotics will have already been distributed to patients who did not need antibiotics as well as inaccurately to treat a multidrug-resistant infection. Whereas culture methods grow isolates from the infection to determine resistance and susceptibility information, which is time-intensive, the single pot NAAT of the present invention with the novel primers and primer combinations can be used to investigate the presence of genes or single nucleotide (SNPs) of these six ESKAPE pathogens as well as others in order to quickly make this determination of whether or not any of the target nucleic acids of the target pathogens are present in the sample.

The PCR protocol can be performed with any type of PCR device. For example, a simple cartridge and instrumentation for rapid thermocycling and detection can be provided. A PCR device cartridge can be used to rapidly thermocycle and detect targets. A cartridge comprised of a 1 mm thick polycarbonate frame with a 0.1 mm film attached to both sides of the frame can be used to maximize heat transfer. The instrumentation can include a 3D printed frame that holds two Peltier thermoelectric elements such that the cartridge may be inserted between them for rapid thermocycling. A fluorescence detector can be mounted on the 3D printed frame such that the fluorescently labeled probes within the cartridge may be excited and emitted light read through the wall of the polycarbonate frame. These devices will be controlled by an microcontroller.

In some embodiments, the PCR cartridge can include two thin films on both sides of a plastic frame. The polycarbonate frame can be created by injection molding, 3D printing, or CNC machining. After which polycarbonate film, 0.025 mm in thickness, can be laminated to the frame at 150° F. using a commercial laminator. The cartridge can be sandwiched between two Peltier Mini Modules from Custom Thermoelectric with a 5.4 W maximum and 6.0 mm×8.2 mm footprint. These thermoelectric elements enable rapid heating and cooling of the cartridge providing a platform for fast amplification. The Peltiers will be controlled by an Motor Shield connector to an microcontroller. The motor shield has two channels, one for each Peltier, and is rated at 2A per channel. This strategy enables extremely fast heating from 65° C. to 95° C. in approximately 1 second based on past experience. Additionally, the Arduino will control the thermistor to read temperature during thermocycling and a small pump from Dolomite microfluidics to apply pressure to the solution in the cartridge such that the film flexes against the Peltier heaters ensuring good thermal contact.

The PCR system can perform rapid amplification and detection of the ESKAPE pathogens via pathogen nucleic acid specific detection, or other target nucleic acids, in less than or about 30 minutes. The instrument and assay can be operated using a bacterial DNA mixed with a PCR solution to create a 20-ul solution. This small volume alongside the Peltier heaters can be amplification and the melt curve process to occur in 30 minutes or less. The sensitivity and specificity of the assay can be determined for each target nucleic acid (e.g., each pathogen species).

In some embodiments, the novel primers and primer combinations used in the PCR amplification and melt-based detection method can be performed so rapidly that the assay can be used in point of care (POC). That is, the system and methods can be used in a clinical setting where the patient provides the urine sample and then receives the results before they leave, which can be within 60 minutes of providing the sample, within 50 minutes, within 45 minutes, within 40 minutes, within 35 minutes, or within 30 minutes. Therefore, the system and method can be implemented in a point of care seeing. The introduction of a point of care assay allows for timely verification of a UTI, thereby enabling: 1) a targeted antibiotic treatment to improve mortality rate: and 2) reducing the overuse of antibiotics. Specifically, the present invention can be used in primary healthcare settings, to provide a rapid (<30 minutes) and accurate POC assay for UTIs. The POC feasibility allows for providing a proper prescription for the correct type of antibiotic(s) and the correct dosing regimen thereof for the detected bacteria pathogens.

Currently, due to the lack of such a diagnostic, generalized prescription of first-line antibiotics for uncomplicated UTIs without a urine culture is an acceptable management strategy. However, antibiotics that are prescribed based on presentation alone results in an error rate of approximately 33%. A POC diagnostic that confirms the presence of a UTI within X-minutes (e.g., 40 minutes), the time frame women are willing to wait in an outpatient facility for test results, will drastically reduce the error rate. Further, hospitals can use a rapid assay that can determine if a nosocomial infection has multidrug resistance.

The implementation of a point-of-care test, which identifies common bacteria Escherichia coli, Klebsiella pneumoniae, and Pseudomonas aeruginosa as well as identifies genetic markers of antibiotic resistance within 60 to 30 minutes can provide not only validation of infection but provide the guidance to narrow the antimicrobial spectrum far more rapidly than traditional methods. The novel single pot NAAT assay and instrumentation described herein provides a point-of-care diagnostic to 1) aid in antimicrobial stewardship by cutting down on the misuse of antibiotics without evidence and 2) provide actionable information regarding the antimicrobial resistance profile of bacterial infections.

The present PCR based assay can be configured for the identification of bacteria or other biomarkers suitable for POC instrumentation. The identification of the target nucleic acids is performed by creating a primer that incorporates melt ‘keys’ sequences into the amplicon through an inner primer specific to each target. The forward target primer includes in order: 3′-a forward species specific region; a melting key region; and a forward super primer region. The reverse target primer includes in order: 3′-a reverse species specific region; and a reverse super primer region. The sequences for the forward target primer and reverse target primer are broken into parts, meaning the common primer, melt-key and target primer are placed together in what can be called the forward and reverse constructs. The definition of a forward construct would be 5′-Common primer+melt-key+target primer-3′. The forward target primer includes in order: 3′-a forward species specific region (target primer); a melting key region; and a forward super primer region (common primer). While the reverse construct is 5′-common primer+target primer-3′. The reverse target primer includes in order: 3′-a reverse species specific region (target primer); and a reverse super primer region (common primer). However, there is nothing preventing the melt key from being placed on the reverse construct instead of the forward construct for the primer pair. In some aspects, both the forward construct and the reverse construct can include the melt key.

The forward species specific region includes a sequence that is specific for the particular target bacteria. That is, the forward species specific region includes a nucleotide sequence that selectively hybridizes to only the nucleic acid of the target bacteria, and thereby is complementary to a unique nucleic acid sequence of that target bacteria.

The melting key region includes a sequence that is configured to produce a complementary sequence in an amplicon have a specific melting temperature with respect to a melting probe. The amplicon from the melting key sequence has a sequence that is substantially complementary with the sequence of a melt probe. This allows a temperature ramp to be performed to see if there is a melting at a defined temperature point of the specific melt key sequence and the general melt probe. It is noted that each target species can have a unique melt key so that its complement hybridizes with the melt probe, with one or more mismatches. These mismatches between the complement sequence of the species specific melt key sequence and the melt probe provide defined melting temperatures. The teach target species can have a its own melt key sequence and thereby the corresponding complement nucleic acid in the amplicon has its own melting point. This allows for multiple species to be detected in the same batch by multiplexing.

The forward super primer region can sometimes be referred to as an excess super primer, such as in the incorporated provisional reference. The forward super primer region includes a sequence that is identical with a forward super primer. The forward super primer region can include a sequence that is unique and not in any of the target species, or it can include a sequence that is common to all target species. The forward super primer region provides for hybridizing so that the forward super primer can be used to amplify and generate a sequence complementary to the forward super primer region and melting key region and forward species specific region. It should be clear that the sequence of the forward super primer exists both as a free primers as well as in the construct. Both super primers form a primer pair as well as both constructs essentially forming a primer pair. The sequence of the forward super primer is also exogenous to the target organisms or target nucleic acids. The sequence of the forward super primer is only common to the amplicon targets because it is designed to be.

The reverse species specific region includes a sequence that is specific for the particular target bacteria. That is, the reverse species specific region includes a nucleotide sequence that selectively hybridizes to only the nucleic acid of the target bacteria, and thereby is complementary to a unique nucleic acid sequence of that target bacteria.

The reverse super primer region can sometimes be referred to as a limiting super primer, such as in the incorporated provisional reference. The reverse super primer region includes a sequence that is identical with a reverse super primer. The reverse super primer region can include a sequence that is unique and not in any of the target species, or it can include a sequence that is common to all target species. The reverse super primer region provides for a resulting amplicon with a complementary region so that the reverse super primer can hybridize therewith to amplify and generate a sequence complementary to the reverse super primer region. It should be clear that the sequence of the reverse super primer exists both as a free primer as well as in the reverse construct. Both super primers form a primer pair as well as both constructs essentially forming a primer pair. The sequence of the reverse super primer is also exogenous to the target organisms or target nucleic acids. The sequence of the reverse super primer is only common to the amplicon targets because it is designed to be.

The multiplexed PCR can be performed by obtaining a sample (e.g., urine sample) and introducing it into a PCR reaction chamber with the multiplex PCR reaction composition. The multiplex PCR reaction composition includes: a forward target primer for each species; a reverse target primer for each species; a forward super primer; a reverse super primer; and a melt probe. In some embodiments, nesting can further include outer primers in the multiplex PCR reaction composition, where the outer primers include the forward outer primer and reverse outer primer that are common to all of the targets as described herein. The multiplexing reaction can then be performed to amplify the amplicons from the primer sets. The cycles result in the amplicons having the complement of the melt key region sequence for the species that is present in the sample. That is, when a species is present in the sample, the amplicon with the complement of the species specific melt key is present. The nucleic acid amplicon with the compliment of the melt key region sequence is then hybridized with the melt probe, and a temperature ramp can identify the different melting temperatures between any one or more of the amplified nucleic acid with the complement of the melt key region sequences with the melt probe. This is because each complement of the melting key region sequence has a unique melting temperature, and thereby the presence of the melting temperature shows the presence of the target species. Those species not present will not show a melting temperature at the defined melting point for that complement of the species specific melt key region sequence.

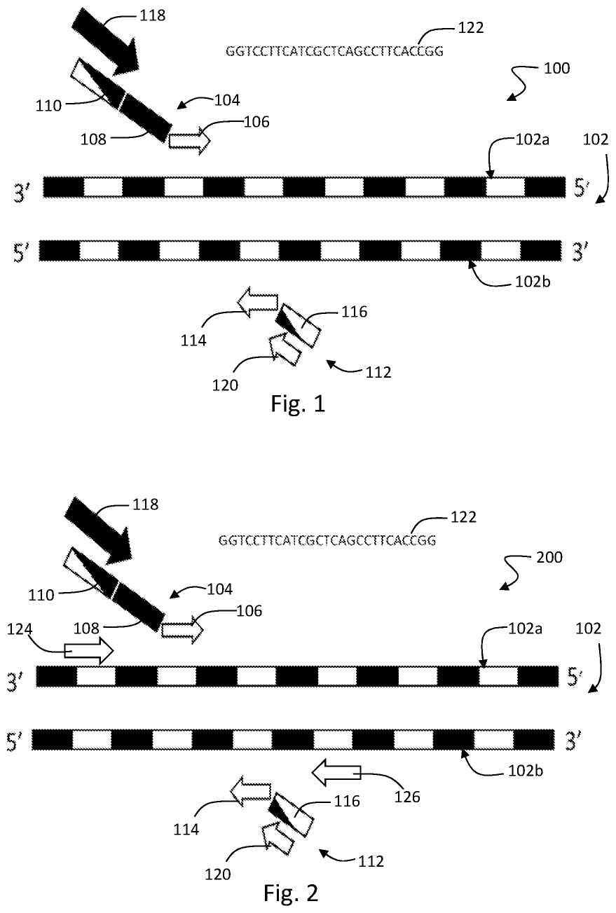

FIG. 1 shows an example of a multiplexing system and method 100. As shown, the target nucleic acid 102 includes to strands, such as an arbitrarily names forward strand 102a and a reverse strand 102b. A forward target primer 104 is provided that includes in order: a forward species specific region 106; a melting key region 108; and a forward super primer region 110. A reverse target primer 112 is provided that includes in order: a reverse species specific region 114; and a reverse super primer region 116. The forward target primer 104 and reverse target primer 112 are a target primer pair that is specific for each target bacteria or other nucleic acid source target. A forward super primer 118 is also provided that hybridizes with the forward super primer region 110. A reverse super primer 120 is also provided that hybridizes with the reverse super primer region. The forward super primer and reverse super primer form a common primer pair. A melting probe 122 is also provided that hybridizes with and has a different melting temperature with each melt key sequence of each forward target primer (or reverse target primer in some embodiments).

The melt probe is a sequence that has high identity (e.g., almost identical sequences) with each of the species specific melt keys, but with at least one nucleotide mismatch. The melt keys region sequences are designed to have a complement thereof with a specific melting temperature with the melt probe. The melt probe can include a label for visualization, such as fluorescence. The melt probe can have a fluorescent label at one end and a quenching agent at the other. Any labeling or identification markers can be applied to the melting probe so that the temperature ramp assay can be performed to find the different melting temperatures that are present in the amplified sample.

Optimization of a multiplexed PCR assay is notoriously difficult requiring careful consideration and testing of primers, buffer, dNTPs, enzyme, and MgCl concentrations for each set of primer combinations. To alleviate this issue and enable a high degree of multiplexing a nested PCR strategy can be performed that uses low concentrations of inner primers that are the forward target primer and reverse target primers and outer primers that are a forward universal primer (e.g., common to 16S region target of all species) and a reverse universal primer (e.g., common to 16S region target of all species). The nested PCR strategy also uses the forward super primer and the reverse super primer, which are a super primer pair common to all targets. This strategy simplifies the creation of a highly multiplexed assay as the low concentration of primer specific to each target proves far more forgiving than a traditional multiplexed assay. Further, using ‘extreme PCR’ like conditions and small volumes can dramatically decrease the time from sample-to-answer to under 30 minutes. Details of these innovative and novel approaches are provided below.

FIG. 2 shows an example of a nested multiplexing system and method 200. As shown, the target nucleic acid 102 includes to strands, such as an arbitrarily names forward strand 102a and a reverse strand 102b. A forward target primer 104 is provided that includes in order: a forward species specific region 106; a melting key region 108; and a forward super primer region 110. A reverse target primer 112 is provided that includes in order: a reverse species specific region 114; and a reverse super primer region 116. The forward target primer 104 and reverse target primer 112 are a target primer pair that is specific for each target bacteria or other nucleic acid source target. A forward super primer 118 is also provided that hybridizes with the forward super primer region 110. A reverse super primer 120 is also provided that hybridizes with the reverse super primer region. The forward super primer and reverse super primer form a common primer pair. A melting probe 122 is also provided that hybridizes with and has a different melting temperature with each melt key sequence of each forward target primer (or reverse target primer in some embodiments). The forward target primer and reverse target primer are considered to be the inner primer pair for a nested PCR. Additionally, the nested PCR includes a forward universal primer 124 that is common to all of the target nucleic acids (e.g., common to 16s region of bacteria) and a reverse universal primer 126 that is common to all of the target nucleic acids (e.g., common 16s region) to form a universal primer pair.

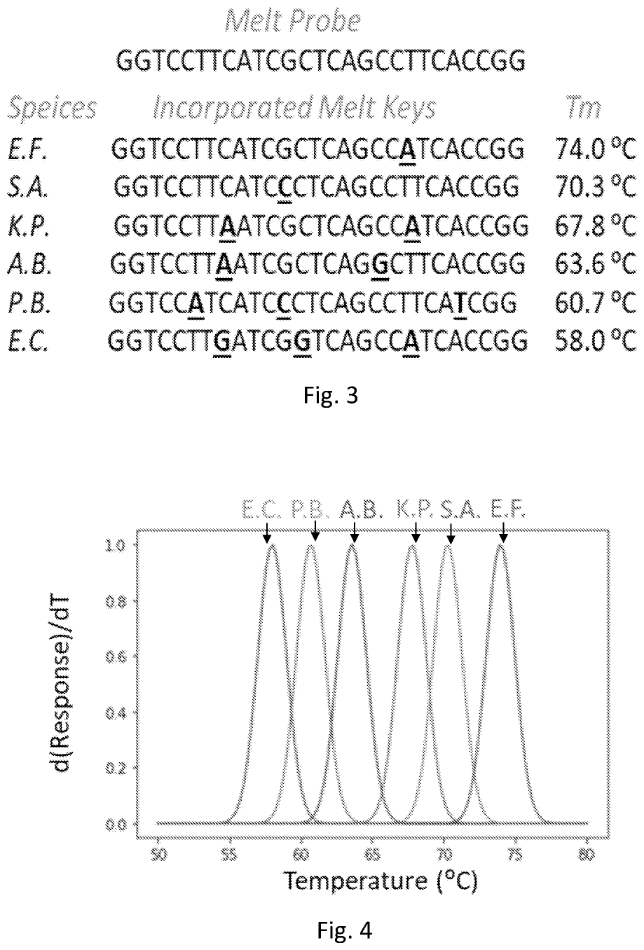

FIG. 3 shows an example melt probe and six melt key region sequences that are included in the forward target primer (or reverse target primer). However, it should be realized that the melt probe can have any sequence that does not hybridize with a nucleic acid of a target. The melt probe can have a unique sequence so that the melt keys can be generated with mismatches (e.g., similar to SNP compared to melt probe). The species designation is arbitrary so long as it is coupled to the target specific region. For example, while the E.F. melt key is 74° C., it could be swapped with the E.C. melt key to have a 58.0° C. melting temperature (Tm). The SEQ ID Nos for each sequence in FIG. 3 is provided in Table 2.

FIG. 4 shows the melting temperature as a peak for each of the targets. As such, the melting temperatures of FIG. 3 are shown by the fluorescent at the temperature to define the melting temperature (Tm). As such, a peak at 58° C. indicates E.C. is present, a peak at 60.7° C. indicates P.B. is present, a peak at 63.6° C. indicates A.B. is present, a peak at 67.8° C. indicates K.P. is present, a peak at 70.3° C. indicates S.A. is present, and a peak at 74.0° C. indicates E.F. is present. The absence of any one or more peaks in the Tm analysis indicates that the corresponding target bacteria is absence.

The melt curve analysis is performed by precisely ramping the temperature within the cartridge and monitoring the fluorescence from the melt probe. For example, a Qiagen OEM ESElog fluorescence detector can be used to monitor the change in fluorescence as the probes anneal from the amplicons. This OEM ESElog fluorescence detector is also controlled by the Arduino. A single channel fluorescence detector can excite the fluorescent molecules within the chamber through the transparent walls of the cartridge and read the emitted light. A program is created within an IDE to first thermocycle the PCR mixture for up to 50 cycles followed by a melt curve analysis from 55° C.-80° C.

Table 2 shows the Melt Probe and Melt Keys with the corresponding SEQ ID NO.

TABLE 2
Melt Probe and Keys

| Melt | 5′-GGTCCTTCATCGCTCAGCCTTCACCGG | Melt |
|---|---|---|
| Probe | SEQ ID NO: 1 | Temp |
| Melt | 5′-GGTCCTTCATCGCTCAGCC [symbol] TCACCGG-3′ | 74.0 C. |
| Key 1 | SEQ ID NO: 2 | |
| Melt | 5′-GGTCCTTCATC [symbol] CTCAGCCTTCACCGG-3′ | 70.3 C. |
| Key 2 | SEQ ID NO: 3 | |
| Melt | 5′-GGTCCTT [symbol] ATCGCTCAGCC [symbol] TCACCGG-3′ | 67.8 C. |
| Key 3 | SEQ ID NO: 4 | |
| Melt | 5′-GGTCCTT [symbol] ATCGCTCAG [symbol] CTTCACCGG-3′ | 63.6 C. |
| Key 4 | SEQ ID NO: 5 | |
| Melt | 5′-GGTCC [symbol] TCATC [symbol] CTCAGCCTTCA [symbol] CGG-3′ | 60.7 C. |
| Key 5 | SEQ ID NO: 6 | |
| Melt | 5′-GGTCCTT [symbol] ATCG [symbol] TCAGCC [symbol] TCACCGG-3′ | 58.0 C. |
| Key 6 | SEQ ID NO: 7 | |

The forward target primer includes in order: 3′-a forward species specific region (target primer); a melting key region; and a forward super primer region (common primer). The forward species specific region can be any target specific sequence. The melting key region can be any of the melting key regions of Table 2. The forward super primer region can be referred to as the forward common primer of Table 3. The reverse target primer includes in order: 3′-a reverse species specific region (target primer); and a reverse super primer region (common primer). The reverse species specific region can be any target specific sequence. the reverse supper primer region (common primer) can be the sequence in Table 3. The sequences in Table 3 are also the forward super primer and reverse super primer that are the primers for the amplicon. The super primer sequences are identical to those in the forward and reverse constructs. This occurs because the super primer binds to the opposite strand once incorporated into the amplicon. It also prevents any amplification until the construct is incorporated then amplified.

TABLE 3
Common Primers (Super Primer Region)

| | | |
|---|---|---|
| Forward Common Primer | 5′-GCCCAGCAAGATCCAATCTCA-3′ | 57.4° C. |
| (Forward Super Primer | SEQ ID NO: 8 | |
| Region) | | |
| Reverse Common Primer | 5′-GTGGCAGGGCGCTACGAACAAT-3′ | 62.5° C. |
| (Reverse Supper Primer | SEQ ID NO: 9 | |
| Region) | | |

In view of Tables 2 and 3, the forward target primer (forward construct) can be prepared as defined when the forward target specific region sequence is determined, and the reverse target primer (reverse construct) can be prepared as defined when the reverse target specific region sequence is determined.

In some embodiments, the forward species specific region for the first target specific primer and the reverse species specific region for the reverse target specific primer for the six bacteria species is provided in Table 4. However, it should be recognized that different sequences could be used for the target specific regions.

TABLE 3
Species-Specific Primers for ESKAPE pathogens

| S | Forward | Reverse |
|---|---|---|
| E.F. | 5′-GAAAAAACAATAGAAGAATTAT -3 | 5- TGCTTTTTTGAATTCTTCTTTA-3 |
| | SEQ ID NO: 10 | SEQ ID NO: 11 |
| S.A. | 5′-GTCGGTACACGATATTCTTCACG-3 | 5′- |
| | SEQ ID NO: 12 | GAGATTTCAGTAGATAATACAACA- |
| | | 3′ |
| | | SEQ ID NO: 13 |
| KP. | 5′-ATTTGAAGAGGTTGCAAACGAT-3 ′ | 5′ - |
| | SEQ ID NO: 14 | TTCACTCTGAAGTTTTCTTGTGTTC- |
| | | 3′ |
| | | SEQ ID NO: 15 |
| A.B. | 5′-TAATGCTTTGATCGGCCTTG-3′ | 5′- TGGATTGCACTTCATCTTGG-3′ |
| | SEQ ID NO: 16 | SEQ ID NO: 17 |
| P.A. | 5′- | 5′- CTTGCGGCTGGCTTTTTCCAG-3′ |
| | ATGAACAACGTTCTGAAATTCTCTGCT-3′ | SEQ ID NO: 19 |
| | SEQ ID NO: 18 | |
| E.C. | 5′-TATGGAATTTCGCCGATTTT-3′ | 5′ -TGTTTGCCTCCCTGCTGCGG-3′ |
| | SEQ ID NO: 20 | SEQ ID NO: 21 |

Full Nucleic Acid Sequences for the Forward Construct ESKAPE Pathogens are provided below, where the melt key region sequence is underlined.

E. faecium w/ Melt Key 1 -
SEQ ID NO: 22
5′ -GCCCAGCAAGATCCAATCTCAGGTCCTTCATCGCTCAGCCATCAC
CGGGAAAAAACAATAGAAGAATTAT-3′
S. aureus w/ Melt Key 2 -
SEQ ID NO: 24
5′- GCCCAGCAAGATCCAATCTCAGGTCCTTCATCCCTCAGCCTTCAC
CGGGTCGGTACACGATATTCTTCACG-3′
K. pneumoniae w/ Melt Key 3 -
SEQ ID NO: 26
5′- GCCCAGCAAGATCCAATCTCAGGTCCTTAATCGCTCAGCCATCAC
CGGATTTGAAGAGGTTGCAAACGAT-3′
A. baumannii w/ Melt Key 4 -
SEQ ID NO: 28
5′- GCCCAGCAAGATCCAATCTCAGGTCCTTAATCGCTCAGGCTTCAC
CGGTAATGCTTTGATCGGCCTTG-3′
P. aeruginosa w/ Melt Key 5 -
SEQ ID NO: 30
5′- GCCCAGCAAGATCCAATCTCAGGTCCATCATCCCTCAGCCTTCAT
CGGATGAACAACGTTCTGAAATTCTCTGCT-3′
E. coli w/ Melt Key 6 -
SEQ ID NO: 32
5′- GCCCAGCAAGATCCAATCTCAGGTCCTTGATCGGTCAGCCATCAC
CGGTATGGAATTTCGCCGATTTT-3′

Full Nucleic Acid Sequences for the Reverse Constructs ESKAPE Pathogens are provided below, with form primer pairs with the Forward Constructs provided above.

E. faecium -
SEQ ID NO: 23
5′ -GTGGCAGGGCGCTACGAACAATTGCTTTTTTGAATTCTTCT
TTA-3′
S. aureus -
SEQ ID NO: 25
5′ -GTGGCAGGGCGCTACGAACAATGAGATTTCAGTAGATAAT
ACAACA-3′
K. pneumoniae -
SEQ ID NO: 27
5′-GTGGCAGGGCGCTACGAACAATTTCACTCTGAAGTTTTCTTGT
GTTC-3′
A. baumannii -
SEQ ID NO: 29
5′ -GTGGCAGGGCGCTACGAACAATTGGATTGCACTTCATCTTGG-3′
P. aeruginosa -
SEQ ID NO: 31
5′-GTGGCAGGGCGCTACGAACAATCTTGCGGCTGGCTTTTTCCAG-3′
E. coli -
SEQ ID NO: 33
5′-GTGGCAGGGCGCTACGAACAATTGTTTGCCTCCCTGCTGCGG-3

In some embodiments, the PCR compositions can include outer primers where the forward construct and reverse construct then are considered to be inner primers. The outer primers can include those identified in Table 4.

TABLE 4
Outer Primers

| Gene | Forward | Reverse |
|---|---|---|
| GyrA | TTGCGCCATACGGACGAT | GCGACGTCATCGGTA-AATACCA |
| Outer | SEQ ID NO: 34 | SEQ ID NO: 35 |
| ParC | TGAGCCATGCGCACCAT | GGCGAGATTTTGGGTAAATACCA |
| Outer | SEQ ID NO: 36 | SEQ ID NO: 37 |

In some embodiments, primers, such as the outer primers can include single nucleotide polymorphisms (SNPs), which are distinguished in the GryA and ParC Genes. The SNPs are targeted on the Forward Primer. The SNPs from a gene share the outer primers. Table 5 shows the target specific primers for constructs or outer primers

TABLE 5
Target Specific Primer for Construct

| Gene | Forward Primer | Reverse Primer |
|---|---|---|
| GyrA | CCCCCATGGCGAGAAA | GTTAGGCAGACGGCTTGG |
| C261A | SEQ ID NO: 38 | TARAATA |
| | | SEQ ID NO: 39 |
| GyrA | CCCCCATGGCGAGAAG | GTTAGGCAGACGGCTTGGT |
| C261G | SEQ ID NO: 40 | ARAATA |
| | | SEQ ID NO: 41 |
| ParC | CGGAAAACGCCTACTTAAACTA | GTGCCGTTAAGCAAAATGT |
| T80A | SEQ ID NO: 42 | SEQ ID NO: 43 |
| ParC | CGGAAAACGCCTACTTAAACTI | GTGCCGTTAAGCAAAATGT |
| T80T | SEQ ID NO: 44 | SEQ ID NO: 45 |

Some examples of the corresponding forward constructs can include (where the melt key region sequence is underlined):

GyrA C261A -
SEQ ID NO: 46
5′- GCCCAGCAAGATCCAATCTCAGGTCCTTCATCGCTCAGCCATCA
CCGGCCCCCATGGCGAGAAA-3′
GyrA C261G -
SEQ ID NO: 48
5′- GCCCAGCAAGATCCAATCTCAGGTCCTTCATCCCTCAGCCTTCA
CCGGCCCCCATGGCGAGAAG-3′
ParC T80A -
SEQ ID NO: 50
5′- GCCCAGCAAGATCCAATCTCAGGTCCTTAATCGCTCAGCCATCA
CCGGCGGAAAACGCCTACTTAAACTA- 3′
ParC T80T -
SEQ ID NO: 52
5′- GCCCAGCAAGATCCAATCTCAGGTCCTTAATCGCTCAGGCTTCA
CCGGCGGAAAACGCCTACTIAAACTT-3′

Some examples of the corresponding reverse constructs can include:

GyrA C261A -
SEQ ID NO: 47
5′- GTGGCAGGGCGCTACGAACAATGTTAGGCAGACGGCTTGGTA
RAATA
GyrA C261G -
SEQ ID NO: 49
5′- GTGGCAGGGCGCTACGAACAATGTTAGGCAGACGGCTTGGTA
RAATA
ParC T80A - -
SEQ ID NO: 51
5′- GTGGCAGGGCGCTACGAACAATGTGCCGTTAAGCAAAATGT
ParC T80T -
SEQ ID NO: 53
5′- GTGGCAGGGCGCTACGAACAATGTGCCGTTAAGCAAAATGT

In some embodiments, the detection of Ciproflaxin susceptible bacteria can be performed as it is both prevalent in 17.1% of infections and commonly used to treat UTIs. Specifically, allele-specific primers can be used to investigate the Ser83Leu and Asp87Asn mutations in the gyrA gene, and Ser80Arg and Glu84Val in the parC gene which is informative of resistance to fluoroquinolones such as Ciproflaxin in E. coli as well as the ESKAPE pathogens. The outer primers that target the gyrA and parC genes and inner primers to target the region where the polymorphisms exist are provided herein.

The primers and the melt probe described herein can be used in a one-pot nucleic acid amplification test (NAAT) with a nested PCR process to incorporate pre-determined nucleic acid sequences into a target sequence for a subsequent melt-based detection. After amplification, a melting assay to determine the presence of melting temperature (peaks) is performed. The melt curve analysis distinguishes targets through the dissociation characteristics of double-stranded DNA during heating, wherein the double-stranded DNA is the melt probe hybridized with a melt key region. As the temperature is raised, the double-strand begins to dissociate leading to a change in the absorbance or fluorescence intensity. The temperature at which 50% of DNA is denatured is known as the melting temperature. Typically, the natural sequence of the amplicon is used to distinguish the target. However, this severely limits identification as the naturally occurring sequences often have similar melt temperatures and profiles.

In some embodiments, the incorporation of exogenous nucleic acid sequences, which are the melt key sequences, with known melt temperatures when its complement (e.g., full complementarity) hybridized with the melt probe into the construct allows for the melt temperature analysis. The melt key region sequences becomes incorporated into an amplicon, which enables differentiation via the melt curve analysis. This melt-based technique increases the multiplexing capability of traditional fluorescent-based NAAT through distinguishing targets via the melt-temperature of the incorporated sequence as well as by fluorescent signal. For example, the identified six targets may be distinguished through the incorporation of exogenous sequences (e.g., melt key sequences) with the following melt temperatures: 58.0° C., 60.7° C., 63.6° C., 67.8° C., 70.3° C., and 74.0° C. These six exogenous sequences may have a single shared probe with a fluorescent tag, six different probes with the same fluorescent tag, or any combination therein. While this probe(s) has the same color and would typically be indistinguishable, a melt curve analysis is able to differentiation the target which is present. This melt key is sandwiched between an exogenous forward common primer and an endogenous forward target primer. The target primer hybridizes to the target during the annealing phase of PCR then serves as the starting material for the extension phase. After the extension, the compliment of the extended DNA sequence now contains the compliment of the incorporated melt key and forward primer. As this process is repeated, the amplified material will contain the melt keys. As the exogenous common primers are incorporated into the amplicons, the common primers will begin to amplify the reaction.

However, additional melt key region sequences can be prepared for the melt probe provided herein or other melt probes. Melt probes with other sequences can be generated so that melt key region sequences having different melting temperatures can be generated. Thus, using the principles of the melt probe and the identified melt key region sequences, other melt probe sequences can be determined and the corresponding other melt key region sequences can be determined.

In some embodiments, this one-pot strategy utilizing fluorescence can be used with digital PCR (dPCR) or digital droplet PCR (ddPCR) in addition to a typical PCR.

Typically, NAAT which incorporates exogenous sequences does so through a strategy that requires more than one-pot amplification. This strategy allows for the incorporation of the exogenous sequence into the target, subsequent amplification of that target and fluorescence detection within the same vesicle. Therefore, this reaction may be completed in a droplet format.

The inner primers are selected to target the species-specific variable 16S regions using dendrograms to determine the highest variability regions in the 16S rDNA for the bacteria in this assay. Proprietary python-based software has been developed to generate 16S amplicons from outer primers using 16S sequencing data acquired from Nucleotide Blast. The generated amplicons are then aligned and compared visually using dendrograms. This strategy allows for a narrowing of the inner primer development to a specific variable region of the 16S region. Moreover, this allows the process to minimize both the number of primers in solution as well as the length of the amplicons. The super primers for LATE-PCR, melt probes and universal outer primers targeting the conserved 16S regions can be utilized and are described in the tables herein. The concentration of the inner and optional outer primers can be tuned to achieve similar results between these two assays.

Rapid amplification and detection of targets in less than 30 minutes is based on a fast thermocycling procedure and melt curve analysis (MCA). The MCA can involve a 1 min denaturation at 95° C., hybridization for 1 min at 55° C., and a stepwise temperature increase (0.5° C. per 1 s) from 55° C. to 80° C., therefore, taking 2 minutes and 50 seconds in full. There can be a 5-minute sample preparation step prior to thermocycling 22 minutes and 10 seconds remaining for the PCR amplification. The 50-cycle PCR given the assumption temperature ramping takes 3 seconds and initial denaturation at 95° C. for 1 min can have a cycle time of 23.6 seconds at most. By increasing the dNTPs and polymerase, as described in Table 4, the 15s extension time can be reduced to 5s if the amplicon is kept short enough thereby achieving the sub-23-second goal. Further reduction of the denaturation and annealing times by altering the primer and magnesium concentration can be achieved.

TABLE 6
Concentration Alterations for Extreme PCR

| Primer/Probe Description | Concentration | |
|---|---|---|
| Forward Common Primer | 0.015-4.000 | μM |
| Forward Reverse Primer | 0.015-4.000 | μM |
| Forward Construct | 0.02 μM or 0.2 μM | |
| Reverse Construct | 0.02 μM to 0.2 μM | |
| Fluorogenic Melt Probe | 0.001 μM to 0.1 μM | |
| dNTPs | 1.000-2.500 | mM |
| Polymerase | 1-10 | U/Rxn |
| Magnesium Chloride | 1 mM-4.5 | mM |

| | |
|---|---|
| Unknown DNA sample | variable |

In some embodiments, the extension time of the assay can be reduced to 5 seconds or less. The denaturation and annealing times are ideally be reduced to 2 and 5 seconds respectively.

Procedure for Multiplexed PCR

Step 1. Prepare a PCR solution to achieve concentration within Table 6 accounting for the additional volume of the DNA sample.

Step 2. Add the unknown DNA sample to the PCR solution of Step 1.

Step 3. Place sample in a thermocycler.

Step 4. Perform PCR: The PCR may consist of 3-step or 2-step PCR. 3-step PCR consists of N cycles of denature-anneal-extend, where the denature step typically occurs at 95 C, the extend step at 72 C and the annealing step between 55-65 C. Where each step is typically 1 minute or less. In this case, the N cycles may be run each cycle has identical conditions. The 3-step PCR may also be run where the annealing temperature are lower in the initial steps with longer time periods. The annealing temperature monotonically increase over with each cycle will the time periods monotonically decrease with each cycle. Similarly, this process can be performed with 2-step PCR. Which consists of a denature step and combined anneal-extend step.

Step 5. Perform Melt Curve analysis: The melt curve analysis is performed by monitoring the fluorescent intensity of the fluorogenic melt probe while increasing the temperature at a given rate between a start and end temperature. The start temperature should be below that of the minimum melt temperature of the melt keys, while the end temperature is above that of the maximum value of the melt keys. In the example above, a start temperate of 55 C ensures melt key 6 would be detected. An end temperature of 75 C ensure Melt Key 1 would be detected. In this example, the temperature could be increased from 55 C to 75 C at a typical rate of 0.1 C/s. However, ramping rates higher or low than 0.1 C/s could be used.

Step 6. The fluorescence data from the melt curve analysis is analyzed for each fluorogenic melt probe. The derivative of the fluorescent signal plotted against temperature will yield one peak per amplicon product from the PCR. If no amplicons are present, the derivative curve will not contain any peaks. The temperature at which these peaks occurs is defined as the melt temperature, which corresponds to the designed melt temperature of the melt probe. The melt temperatures present in the derivation curve indicates which products existed in the unknown DNA sample. This process is repeated for each fluorogenic melt probe.

Procedure for Multiplexed dPCR

The dPCR procedure is similar to the above procedure.

Step 1. Prepare a PCR solution to achieve concentration within Table 4 accounting for the additional volume of the DNA sample.

Step 2. Add the unknown DNA sample to the PCR solution of Step 1.

Step 3. The PCR solution is broken into droplets, either will a microfluidic digital droplet platform where the droplets are suspended free in an oil solution or a dPCR microfluidic chip where the droplets are trapped within micro-, nano- or pico-cavities and separated with an oil solution.

Step 4. Same as above for Multiplexed PCR, except in a dPCR format.

Step 5. Same as above for Multiplexed PCR, expect each droplet must be analyzed individually using the melt curve analysis process.

Step 6. Same as above for Multiplexed PCR, expect again for each droplet individually.

In some embodiments, a primer composition for amplifying a target nucleic acid of a plurality of target organisms can include: a first primer having in order: 3′-a first target specific sequence; a melt key region; and a first super primer region, wherein the first target specific sequence includes a nucleic acid sequence that specifically hybridizes with a first unique nucleic acid sequence of a first target organism over nucleic acid sequences of other organisms, the melt key region includes a nucleic acid sequence that has full identity or less than full identity with a melt key probe and a complement of the melt key region hybridizes with the melt key probe with no mismatch or at least one nucleotide mismatch with the melt key probe, and the first super primer region includes a nucleic acid sequence that is exogenous to the plurality of target organisms; and a second primer having in order: 3′-target specific sequence; and a second super primer region, wherein the second target specific sequence includes a nucleic acid sequence that specifically hybridizes with a second unique nucleic acid sequence of the first target organism over nucleic acid sequences of other organisms, and the second super primer region includes a nucleic acid sequence that is exogenous to the plurality of target organisms, wherein the first primer and second primer form a primer pair.

In some embodiments, a primer composition for amplifying at least one target nucleic acid of a plurality of target nucleic acids can include: a first primer having in order: 3′-a first target specific sequence; a melt key region; and a first super primer region, wherein the first target specific sequence includes a nucleic acid sequence that specifically hybridizes with a first unique nucleic acid sequence of a first target nucleic acid over nucleic acid sequences of other nucleic acids, the melt key region includes a nucleic acid sequence that has full identity or less than full identity with a melt key probe and a complement of the melt key region hybridizes with the melt key probe with no mismatch or at least one nucleotide mismatch with the melt key probe, and the first super primer region includes a nucleic acid sequence that is exogenous to the plurality of target nucleic acids; and a second primer having in order: 3′-target specific sequence; and a second super primer region, wherein the second target specific sequence includes a nucleic acid sequence that specifically hybridizes with a second unique nucleic acid sequence of the first target nucleic acid over nucleic acid sequences of other nucleic acids, and the second super primer region includes a nucleic acid sequence that is exogenous to the plurality of target nucleic acids, wherein the first primer and second primer form a primer pair.

In some embodiments, the primer composition can include: a plurality of first primers, wherein each first primer has the first target specific sequence that specifically hybridizes with a first nucleic acid sequence of a respective first target organism over nucleic acid sequences of other target organisms, each first primer has a melt key region that is different from melt key regions of other first primers, and each first primer has the same first super primer region; and a plurality of second primers, wherein each second primer has the second target specific sequence that specifically hybridizes with a second nucleic acid sequence of a respective first target organism over nucleic acid sequences of other target organisms, and each second primer has the same second super primer region.

In some embodiments, the primer composition can include: a plurality of first primers, wherein each first primer has the first target specific sequence that specifically hybridizes with a first nucleic acid sequence of a respective first target nucleic acid over nucleic acid sequences of other target nucleic acids, each first primer has a melt key region that is different from melt key regions of other first primers, and each first primer has the same first super primer region; and a plurality of second primers, wherein each second primer has the second target specific sequence that specifically hybridizes with a second nucleic acid sequence of a respective first target nucleic acid over nucleic acid sequences of other target nucleic acids, and each second primer has the same second super primer region.

In some embodiments, the primer composition can include: a first super primer having the sequence of the first super primer region; and a second super primer having the sequence of the second super primer region.

In some embodiments, the primer composition can include the melt key probe, wherein a complement nucleic acid of each melting key region has a different melting temperature with the melt key probe, wherein each first primer has a different nucleotide mismatch in the melt key region from each other first primer.

In some embodiments, each first primer and second primer are an inner primer pair of a nested primer composition. The primer composition can also include: a first universal primer having a nucleic acid sequence that hybridizes to a first common nucleic acid sequence present in each of the plurality of target organisms; and a second universal primer having a nucleic acid sequence that hybridizes to a second common nucleic acid sequence present in each of the plurality of organisms, wherein the first universal primer and second universal primer are an outer primer pair of the nested primer composition.

In some embodiments, the first super primer region includes the nucleotide sequence of SEQ ID NO: 8 or complement thereof; and the second super primer region includes the nucleotide sequence of SEQ ID NO: 9.

In some embodiments, the plurality of melt key regions of the plurality of first primers includes one nucleotide sequence of SEQ ID NO: 2, SEQ ID NO: 3, SEQ ID NO: 4, SEQ ID NO: 5, SEQ ID NO: 6, and SEQ ID NO: 7, or complement thereof. The melt probe can have the nucleotide sequence of SEQ ID NO: 1 or complement thereof.

In some embodiments, the plurality of melt key regions of the plurality of first primers includes one nucleotide sequence of SEQ ID NO: 2, SEQ ID NO: 3, SEQ ID NO: 4, SEQ ID NO: 5, SEQ ID NO: 6, and SEQ ID NO: 7, or complement thereof; and the melt probe has the nucleotide sequence of SEQ ID NO: 1 or complement thereof.

In some embodiments, the first universal primer has the nucleotide sequence of SEQ ID NO: 34 or SEQ ID NO: 36; and the second universal primer has the nucleotide sequence of SEQ ID NO: 35 or SEQ ID NO: 37.

In some embodiments, each first target specific sequence of the plurality of first primers includes one nucleotide sequence of SEQ ID NO: 10, SEQ ID NO: 12, SEQ ID NO: 14, SEQ ID NO: 16, SEQ ID NO: 18, and SEQ ID NO: 20; and each second target specific sequence of the plurality of second primers includes one nucleotide sequence of SEQ ID NO: 11, SEQ ID NO: 13, SEQ ID NO: 15, SEQ ID NO: 17, SEQ ID NO: 19, and SEQ ID NO: 21.

In some embodiments, the plurality of first primers includes one nucleotide sequence of SEQ ID NO: 22, SEQ ID NO: 24, SEQ ID NO: 26, SEQ ID NO: 28, SEQ ID NO: 30, and SEQ ID NO: 32; and the plurality of second primers includes one nucleotide sequence of SEQ ID NO: 23, SEQ ID NO: 25, SEQ ID NO: 27, SEQ ID NO: 29, SEQ ID NO: 31, and SEQ ID NO: 33.

In some embodiments, a melt-based nucleic acid system can include: a melt probe having a nucleotide sequence; and a plurality of first primers, each first primer having in order: 3′-a first target specific sequence; a melt key region; and a first super primer region, wherein the first target specific sequence includes a nucleic acid sequence that specifically hybridizes with a first unique nucleic acid sequence of a first target organism over nucleic acid sequences of other target organisms, the melt key region includes a nucleic acid sequence that has full identity or less than full identity (e.g., less than full complementarity, such as 1, 2, 3, or more mismatches) with the melt key probe and a complement of the melt key region (e.g., full identity complementarity) hybridizes with the melt key probe with at least one nucleotide mismatch with the melt key probe, wherein each first primer has a melt key region that is different from melt key regions of other first primers, and each first primer has the same first super primer region; and a plurality of primer nucleic acids each having a melt key region with a nucleotide sequence with less than full uniformity (e.g., less than full identify complementarity, such as 1, 2, 3 or more mismatches) compared to the melt probe, wherein each first primer has a different nucleotide mismatch from each other first primer.

In some embodiments, a melt-based nucleic acid system can include: a melt probe having a nucleotide sequence; and a plurality of first primers, each first primer having in order: 3′-a first target specific sequence; a melt key region; and a first super primer region, wherein the first target specific sequence includes a nucleic acid sequence that specifically hybridizes with a first unique nucleic acid sequence of a first target nucleic acid over nucleic acid sequences of other target nucleic acids, the melt key region includes a nucleic acid sequence that has full identity or less than full identity (e.g., less than full complementarity, such as 1, 2, 3, or more mismatches) with the melt key probe and a complement of the melt key region (e.g., full identity complementarity) hybridizes with the melt key probe with no nucleotide mismatch or at least one nucleotide mismatch with the melt key probe, wherein each first primer has a melt key region that is different from melt key regions of other first primers, and each first primer has the same first super primer region; and a plurality of primer nucleic acids each having a melt key region with a nucleotide sequence with less than full uniformity (e.g., less than full identify complementarity, such as 1, 2, 3 or more mismatches) compared to the melt probe, wherein each first primer has a different nucleotide mismatch from each other first primer.

In some embodiments, the melt probe has the nucleotide sequence of SEQ ID NO: 1. In some aspects, the plurality of melt key regions of the plurality of first primers includes one nucleotide sequence of SEQ ID NO: 2, SEQ ID NO: 3, SEQ ID NO: 4, SEQ ID NO: 5, SEQ ID NO: 6, and SEQ ID NO: 7, or complement thereof.

In some embodiments, a method of detecting a plurality of target organisms in a sample can include: providing the sample into a PCR device; providing a PCR composition into the PCR device with the sample; performing PCR to generate a plurality of amplicons, each amplicon including one melt key complement region having a nucleotide sequence with full complementarity to the respective melt key region; performing a melt curve analysis with the plurality of amplicons and the melt key probe;

detecting at least one melting temperature for the melt key probe and at least one amplicon including one melt key region; and determining presence of at least one target organism of the plurality of target organisms by detecting the presence of a predefined melting temperature for the melt key probe and the at least one amplicon including one melt key region.

In some embodiments, a method of detecting a plurality of target nucleic acids in a sample can include: providing the nucleic acid sample into a PCR device; providing a PCR composition into the PCR device with the sample; performing PCR to generate a plurality of amplicons, each amplicon including one melt key complement region having a nucleotide sequence with full complementarity to the respective melt key region; performing a melt curve analysis with the plurality of amplicons and the melt key probe; detecting at least one melting temperature for the melt key probe and at least one amplicon including one melt key region; and determining presence of at least one target nucleic acid of the plurality of target nucleic acids by detecting the presence of a predefined melting temperature for the melt key probe and the at least one amplicon including one melt key region.

In some embodiments, the PCR composition includes a fluorogenic melt probe, the method comprising: determining fluorescence versus temperature; identifying a peak for each predetermined melting temperature; and determining the peak to represent presence of the at least one target nucleic acid/organism of the plurality of target nucleic acid/organisms. In some aspects, each predefined melting temperature is associated with a particular target nucleic acid/organism of the plurality of target nucleic acids/organisms. In some aspects, the PCR is bulk PCR or traditional PCR. In some aspects, the PCR is dPCR or ddPCR. In some aspects, the method includes: plotting a graph of the fluorescence versus temperature; determining a melting temperature for each peak in the graph; and identifying a particular target nucleic acid/organism associated with the melting temperature of a particular peak in the graph.

In some embodiments, the PCR composition can include: a plurality of first primers, each first primer having in order: 3′-a first target specific sequence; a melt key region; and a first super primer region, wherein the first target specific sequence includes a nucleic acid sequence that specifically hybridizes with a first unique nucleic acid sequence of a first target nucleic acid/organism over nucleic acid sequences of other target nucleic acids/organisms, the melt key region includes a nucleic acid sequence that has full identity or less than full identity with a melt key probe and a complement of the melt key region hybridizes with the melt key probe with no nucleotide mismatch or at least one nucleotide mismatch with the melt key probe, and the first super primer region includes a nucleic acid sequence that is exogenous to the plurality of target nucleic acids/organisms, wherein each first primer has a melt key region that is different from melt key regions of other first primers, and each first primer has the same first super primer region; a plurality of second primers, each second primer having in order: 3′-target specific sequence; and a second super primer region, wherein the second target specific sequence includes a nucleic acid sequence that specifically hybridizes with a second nucleic acid sequence of the first target nucleic acid/organism over nucleic acid sequences of other target nucleic acids/organisms, and the second super primer region includes a nucleic acid sequences that is exogenous to the plurality of target nucleic acids/organisms, wherein the first primer and second primer form a primer pair; a first super primer having the sequence of the first super primer region; a second super primer having the sequence of the second super primer region; and the melt key probe, wherein each first primer has a different melting temperature with the melt key probe, wherein each first primer has a different nucleotide mismatch from each other first primer.

One skilled in the art will appreciate that, for the processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references recited herein are incorporated herein by specific reference in their entirety.

### REFERENCES

Multiplex Quantitative Polymerase Chain Reaction In One Reaction (US20180340213A1);

Quantitative multiplex polymerase chain reaction in two reactions (US20180340214A1); Method and kit for primer based multiplex amplification of nucleic acids employing primer binding tags (U.S. Pat. No. 7,851,148B2).

1. A primer composition for amplifying a target nucleic acid of a plurality of target nucleic acids, the primer comprising:
a first primer having in order: 3′-a first target specific sequence; a melt key region; and a first super primer region, wherein the first target specific sequence includes a nucleic acid sequence that specifically hybridizes with a first nucleic acid sequence of a first target nucleic acid over nucleic acid sequences of other nucleic acids, the melt key region includes a nucleic acid sequence that has full identity or less than full identity with a melt key probe and a complement of the melt key region hybridizes with the melt key probe with no nucleotide mismatch or at least one nucleotide mismatch with the melt key probe, and the first super primer region includes a nucleic acid sequence that is exogenous to the plurality of target nucleic acids; and
a second primer having in order: 3′-target specific sequence; and a second super primer region, wherein the second target specific sequence includes a nucleic acid sequence that specifically hybridizes with a second nucleic acid sequence of the first target nucleic acid over nucleic acid sequences of other nucleic acids, and the second super primer region includes a nucleic acid sequence that is exogenous to the plurality of target nucleic acids, wherein the first primer and second primer form a primer pair.

2. The primer composition of claim 1, further comprising:
a plurality of first primers, wherein each first primer has the first target specific sequence that specifically hybridizes with a first nucleic acid sequence of a respective first target nucleic acid over nucleic acid sequences of other target nucleic acids, each first primer has a melt key region that is different from melt key regions of other first primers, and each first primer has the same first super primer region; and
a plurality of second primers, wherein each second primer has the second target specific sequence that specifically hybridizes with a second nucleic acid sequence of a respective first target nucleic acid over nucleic acid sequences of other target nucleic acids, and each second primer has the same second super primer region.

3. The primer composition of claim 2, further comprising:
a first super primer having the sequence of the first super primer region; and
a second super primer having the sequence of the second super primer region.

4. The primer composition of claim 3, further comprising the melt key probe, wherein a complement nucleic acid of each melting key region has a different melting temperature with the melt key probe.

5. The primer composition of claim 4, wherein each first primer and second primer are an inner primer pair of a nested primer composition, further comprising:
a first universal primer having a nucleic acid sequence that hybridizes to a first common nucleic acid sequence present in each of the plurality of target nucleic acids; and
a second universal primer having a nucleic acid sequence that hybridizes to a second common nucleic acid sequence present in each of the plurality of nucleic acids, wherein the first universal primer and second universal primer are an outer primer pair of the nested primer composition.

6. The primer composition of claim 2, wherein:
the first super primer region includes the nucleotide sequence of SEQ ID NO: 8 or complement thereof; and
the second super primer region includes the nucleotide sequence of SEQ ID NO: 9.

7. The primer composition of claim 6, wherein:
the plurality of melt key regions of the plurality of first primers includes one nucleotide sequence of SEQ ID NO: 2, SEQ ID NO: 3, SEQ ID NO: 4, SEQ ID NO: 5, SEQ ID NO: 6, and SEQ ID NO: 7, or complement thereof;
further comprising:
the melt probe having the nucleotide sequence of SEQ ID NO: 1.

8. The primer composition of claim 4, wherein:
the plurality of melt key regions of the plurality of first primers includes one nucleotide sequence of SEQ ID NO: 2, SEQ ID NO: 3, SEQ ID NO: 4, SEQ ID NO: 5, SEQ ID NO: 6, and SEQ ID NO: 7, or complement thereof; and
the melt probe has the nucleotide sequence of SEQ ID NO: 1.

9. The primer composition of claim 5, wherein:
the first universal primer has the nucleotide sequence of SEQ ID NO: 34 or SEQ ID NO: 36; and
the second universal primer has the nucleotide sequence of SEQ ID NO: 35 or SEQ ID NO: 37.

10. The primer composition of claim 2, wherein:
each first target specific sequence of the plurality of first primers includes one nucleotide sequence of SEQ ID NO: 10, SEQ ID NO: 12, SEQ ID NO: 14, SEQ ID NO: 16, SEQ ID NO: 18, and SEQ ID NO: 20; and
each second target specific sequence of the plurality of second primers includes one nucleotide sequence of SEQ ID NO: 11, SEQ ID NO: 13, SEQ ID NO: 15, SEQ ID NO: 17, SEQ ID NO: 19, and SEQ ID NO: 21.

11. The primer composition of claim 2, wherein:
the plurality of first primers includes one nucleotide sequence of SEQ ID NO: 22, SEQ ID NO: 24, SEQ ID NO: 26, SEQ ID NO: 28, SEQ ID NO: 30, and SEQ ID NO: 32; and
the plurality of second primers includes one nucleotide sequence of SEQ ID NO: 23, SEQ ID NO: 25, SEQ ID NO: 27, SEQ ID NO: 29, SEQ ID NO: 31, and SEQ ID NO: 33.

12. A melt-based nucleic acid system, comprising:
a melt probe having a nucleotide sequence; and
a plurality of first primers, each first primer having in order: 3′-a first target specific sequence; a melt key region; and a first super primer region, wherein the first target specific sequence includes a nucleic acid sequence that specifically hybridizes with a first nucleic acid sequence of a first target nucleic acid over nucleic acid sequences of other target nucleic acids, the melt key region includes a nucleic acid sequence that has full identity or less than full identity with the melt key probe and a complement of the melt key region hybridizes with the melt key probe with no nucleotide mismatch or at least one nucleotide mismatch with the melt key probe, wherein each first primer has a melt key region that is different from melt key regions of other first primers, and each first primer has the same first super primer region, and the first super primer region includes a nucleic acid sequence that is exogenous to the plurality of target nucleic acids.

13. The melt-based nucleic acid system of claim 12, wherein the melt probe has the nucleotide sequence of SEQ ID NO: 1.

14. The melt-based nucleic acid system of claim 13, wherein the plurality of melt key regions of the plurality of first primers includes one nucleotide sequence of SEQ ID NO: 2, SEQ ID NO: 3, SEQ ID NO: 4, SEQ ID NO: 5, SEQ ID NO: 6, and SEQ ID NO: 7, or complement thereof.

15. A method of detecting a plurality of target nucleic acids in a sample, the method comprising:
providing the nucleic acid sample into a PCR device;
providing a PCR composition into the PCR device with the sample, the PCR composition comprising:
a plurality of first primers, each first primer having in order: 3′-a first target specific sequence; a melt key region; and a first super primer region, wherein the first target specific sequence includes a nucleic acid sequence that specifically hybridizes with a first nucleic acid sequence of a first target nucleic acid over nucleic acid sequences of other target nucleic acids, the melt key region includes a nucleic acid sequence that has full identity or less than full identity with a melt key probe and a complement of the melt key region hybridizes with the melt key probe with no mismatch or at least one nucleotide mismatch with the melt key probe, and the first super primer region includes a nucleic acid sequence that is exogenous to the plurality of target organisms, wherein each first primer has a melt key region that is different from melt key regions of other first primers, and each first primer has the same first super primer region;
a plurality of second primers, each second primer having in order: 3′-target specific sequence; and a second super primer region, wherein the second target specific sequence includes a nucleic acid sequence that specifically hybridizes with a second nucleic acid sequence of the first target nucleic acid over nucleic acid sequences of other target nucleic acids, and the second super primer region includes a nucleic acid sequences that is exogenous to the plurality of target organisms, wherein the first primer and second primer form a primer pair;
a first super primer having the sequence of the first super primer region;
a second super primer having the sequence of the second super primer region; and
the melt key probe, wherein each first primer has a different melting temperature with the melt key probe, wherein each first primer has a different melt key region sequence from each other first primer;
performing PCR to generate a plurality of amplicons, each amplicon including one melt key complement region having a nucleotide sequence with full complementarity to the respective melt key region;
performing a melt curve analysis with the plurality of amplicons and the melt key probe;
detecting at least one melting temperature for the melt key probe and at least one amplicon including one melt key region; and
determining presence of at least one target nucleic acid of the plurality of target nucleic acids by detecting the presence of a predefined melting temperature for the melt key probe and the at least one amplicon including one melt key region.

16. The method of claim 15, wherein the PCR composition includes a fluorogenic melt probe, the method comprising:
determining fluorescence versus temperature;
identifying a peak for each predetermined melting temperature; and
determining the peak to represent presence of the at least one target nucleic acid of the plurality of target nucleic acids.

17. The method of claim 15, wherein each predefined melting temperature is associated with a particular target nucleic acid of the plurality of target nucleic acids.

18. The method of claim 15, wherein the PCR is not qualitative PCR.

19. The method of claim 15, wherein the PCR is dPCR or ddPCR.

20. The method of claim 16, comprising:
plotting a graph of the fluorescence versus temperature;
determining a melting temperature for each peak in the graph; and
identifying a particular target nucleic acid that is associated with the melting temperature of a particular peak in the graph.
